(19) United States
(12) Patent Application Publication
(10) Pub. No.: US 2018/0126219 A1
(43) Pub. Date: May 10, 2018

(54) METHODS AND APPARATUSES FOR HANDGRIP STRENGTH ASSESSMENT USING PRESSURE-SENSITIVE ELEMENTS

(71) Applicant: KONINKLIJKE PHILIPS N.V., EINDHOVEN (NL)

(72) Inventors: Saman Parvaneh, Malden, MA (US); Mladen Milosevic, Stoneham, MA (US)

(21) Appl. No.: 15/807,840

(22) Filed: Nov. 9, 2017

Related U.S. Application Data
(60) Provisional application No. 62/420,168, filed on Nov. 10, 2016.

(51) Int. Cl. A63B 24/00; A63B 71/06; A61B 5/11; A61B 5/117

(52) U.S. Cl. CPC A63B 24/0062; A63B 24/0075; A63B 71/0622; A61B 5/1125; A61B 5/117; A63B 2225/15; A63B 2220/56; A63B 2220/20; A63B 2225/50; A63B 2220/62; A63B 2024/0068

(57) ABSTRACT

The described embodiments relate to computing devices capable of assessing handgrip strength, and using data collected during a handgrip strength assessment to assist a user or medical provider. The computing device can include an array of pressure sensitive elements to determine a force of pressure applied at different locations on the computing device. The pressure of the handgrip can be monitored for a time period in order to determine metrics such as average strength of the grip over time and decay in strength of the grip over time. Such metrics can be tracked over a multiple handgrip strength assessments in order to track how the metrics change over time. The computing device can communicate with electronic healthcare systems, such as electronic medical records, in order that hospitals and other medical providers can access and analyze the data in order to find clinical pathways to treat related medical conditions.

### CROSS-REFERENCE TO RELATED APPLICATION

The present application claims priority to and benefit of U.S. Provisional Application Ser. No. 62/420,168, filed Nov. 10, 2016, the entirety of which is hereby incorporated by reference herein.

### TECHNICAL FIELD

The present invention is directed generally to health care. More particularly, but not exclusively, various systems, methods, and apparatuses disclosed herein relate to portable computing devices capable of measuring, tracking, and communicating metrics related to handgrip strength.

### BACKGROUND

Many existing medical measurement devices remain strictly mechanical because of their reliability. However, because mechanical measurement devices typically rely on human interpretation, recordation of data provided by such devices can be prone to error. Furthermore, manual recordation of data can disrupt workflow of medical providers and hospital workers. For example, measurements that must be taken from a patient multiple times a day could require a nurse to take the measurements, record the measurements, and later enter the measurements into the patient's medical records. Such a schedule would not provide much available time to take other measurements. Furthermore, even if time was available, certain measurements, such as grip strength, may not be assessed because medical instruments for performing such measurements, such as dynamometers, may be difficult to operate and may tend to slow the workflow of hospital workers.

### SUMMARY

The present disclosure is directed to systems, methods, and apparatuses for assessing grip strength using a portable computing device that is also capable of transmitting grip data to remote computing devices.

Generally, in one aspect, a computing device may include: a pressure sensor comprising an array of sensor elements, the pressure sensor configured to output a signal in response to pressure applied at different locations of the array of sensor elements; a display device, wherein the array of sensor elements are configured to receive the pressure in response to an initial force of pressure applied to the display device, and the signal is output in response to pressure applied at different locations on the display device; one or more processors configured to generate a grip metric based on the signal from the pressure sensor; and a communications interface configured to transfer data corresponding to the grip metric to a remote computing device.

In various embodiments, the grip measurements may include a distance measurement between at least two points of contact at the display device. In various embodiments, the grip measurements include a rate of decay of grip pressure over a period of time when the display device is receiving a variable force of pressure. In various embodiments, the grip metric includes an overall grip strength measurement that is based on a total of individual forces of pressure at the different locations of the array of sensor elements.

In various embodiments, the communications interface may be further configured to receive, from the remote computing device, analytical data based on the grip metric. In various embodiments, the one or more processors are further configured to authenticate a user based on the signal from the pressure sensor.

In another aspect, a method for gathering and communicating grip strength data using a personal computing device may include: by the computing device: generating sensor data when one or more pressures are simultaneously applied to different locations of an exterior surface of the computing device; determining a grip metric based on the sensor data; comparing the grip metric to historical grip metric data accessible to the computing device; and in response to a determination that the grip metric is outside of a threshold tolerance of the historical grip metric data: transmitting data corresponding to the grip metric to a remote computing device. In various embodiments, the method may further include performing a curve fitting operation on the sensor data to identify an exponential function representing the sensor data; wherein grip metric includes a parameter of the exponential function.

The term “controller” is used herein generally to describe various apparatus relating to the operation of one or more pressure sources. A controller can be implemented in numerous ways (e.g., such as with dedicated hardware) to perform various functions discussed herein. A “processor” is one example of a controller, which employs one or more microprocessors that may be programmed using software (e.g., microcode) to perform various functions discussed herein. A controller may be implemented with or without employing a processor, and also may be implemented as a combination of dedicated hardware to perform some functions and a processor (e.g., one or more programmed microprocessors and associated circuitry) to perform other functions. Examples of controller components that may be employed in various embodiments of the present disclosure include, but are not limited to, conventional microprocessors, application specific integrated circuits (ASICs), and field-programmable gate arrays (FPGAs).

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program,” “application,” or “computer program” are used herein in a generic sense to refer to any type of computer code (e.g., software or microcode) that can be employed to program one or more processors or controllers.

The term “addressable” is used herein to refer to a device (e.g., a pressure-based device, a pressure source, a controller or a processor associated with one or more pressure sources, or other non-pressure related devices, etc.) that is configured to receive information (e.g., data) intended for multiple devices, including itself, and to selectively respond to particular information intended for it. The term “addressable” often is used in connection with a networked environment (or a “network,” discussed further below), in which multiple devices are coupled together via some communications medium or media.

In one network implementation, one or more devices coupled to a network may serve as a controller for one or more other devices coupled to the network (e.g., in a master/slave relationship). In another implementation, a networked environment may include one or more dedicated controllers that are configured to control one or more of the devices coupled to the network. Generally, multiple devices coupled to the network each may have access to data that is present on the communications medium or media; however, a given device may be “addressable” in that it is configured to selectively exchange data with (i.e., receive data from and/or transmit data to) the network, based, for example, on one or more particular identifiers (e.g., “addresses”) assigned to it.

The term “network” as used herein refers to any interconnection of two or more devices (including controllers or processors) that facilitates the transport of information (e.g., for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

The term “user interface” as used herein refers to an interface between a human user or operator and one or more devices that enables communication between the user and the device(s). Examples of user interfaces that may be employed in various implementations of the present disclosure include, but are not limited to, switches, potentiometers, buttons, dials, sliders, a mouse, keyboard, keypad, various types of game controllers (e.g., joysticks), track balls, display screens, various types of graphical user interfaces (GUIs), touch screens, microphones and other types of sensors that may receive some form of human-generated stimulus and generate a signal in response thereto.

The term “module” as used herein refers to a hardware or software (or combination of both hardware and software) component of a computing device used to perform a specific task. An example of a “module” used herein is a pressure sensitive touch module, which can communicate with at least one pressure sensor that reads a pressure value at a plurality of touch points during a hand grip strength assessment.

The term “database” as used herein refers to a collection of data and information or data organized in such a way as to allow the data and information or data to be stored, retrieved, updated, and manipulated and to allow them to be presented into one or more formats such as in table form or to be grouped into text, numbers, images, and audio data. The term “database” as used herein may also refer to a portion of a larger database, which in this case forms a type of database within a database. “Database” as used herein also refers to conventional databases that may reside locally or that may be accessed from a remote location, e.g., remote network servers. The database typically resides in computer memory that includes various types of volatile and nonvolatile computer memory. Memory wherein the database resides may include high-speed random access memory or non-volatile memory such as magnetic disk storage devices, optical storage devices, and flash memory. Memory where the database resides may also comprise one or more software for processing and organizing data received by and stored into the database.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIGS. 1A, 1B and 1C illustrate operations that are performed when using a grip strength assessment application according to various embodiments.

FIG. 2 illustrates a system diagram of a computing device that can collect, analyze, and share handgrip data with other computing devices, such as those associated with an electronic healthcare system.

FIG. 3A illustrates a plot of pressure exerted on a computing device, such as any of the devices discussed herein.

FIG. 3B illustrates a plot that includes a fitted exponential curve for determining an exponential equation that best fits the plot of pressure.

FIG. 4 illustrates a method for transmitting handgrip data to a remote computing device, according to some embodiments.

FIG. 5 illustrates a method for comparing grip strength data and transmitting grip strength data based on the comparing, according to some embodiments.

FIG. 6 illustrates a method for generating and comparing coefficients that are derived from grip strength measurement data, according to some embodiments.

### DETAILED DESCRIPTION

The described embodiments relate to systems, methods, and apparatuses for assessing handgrip strength. Handgrip strength can be an indicator of mortality and disability, and provide insight regarding a condition of a patient after surgery. In certain circumstances, handgrip strength assessment can be used to evaluate performance of athletes. Typically, a mechanical dynamometer device is used to measure handgrip strength. However, such devices are typically unable to communicate with electronic healthcare systems, such as databases storing electronic medical records (EMRs). As a result, data regarding handgrip strength is entered manually, which can lead to human error and disruptions in work flow. Additionally, such devices are not typically available outside of a medical office. Consequently, handgrip strength assessments may only be performed infrequently. The embodiments described herein resolve these issues by introducing a portable electronic handgrip strength assessment device that can send and receive data to and from different electronic healthcare systems.

In some embodiments, a grip strength assessment device is set forth as part of a portable electronic device. The portable electronic device may be a wireless communications device already owned/possessed by many users, such as a cell phone, media player, tablet computer, peripheral device, and/or any other portable device that can include a pressure sensor. The portable electronic device can include one or more pressure sensors that can measure the pressure of an overall grip of a human hand, or other body part, on the portable electronic device. Additionally, the one or more pressure sensors can measure the pressure from individual fingers of a human hand. In some embodiments, the pressure sensors are an array of capacitive touch sensors, or other touch-sensitive elements, that can evaluate an amount of pressure applied to one or more surfaces of the portable electronic device. For example, the portable electronic device can include a display panel and the array of capacitive touch sensors can be arranged to evaluate an amount of pressure applied to the display panel.

Data derived from the pressure sensor(s) can be used to make various determinations about a user that is gripping the portable computing device. The portable computing device can include a feature extraction module that uses the data to calculate the overall grip pressure of the user, pressure from each finger pressed against the portable computing device, location of each finger on the portable computing device, and/or distance between fingers. The portable computing device can also include and/or access a grip feature database. The grip feature database can be used to store historical data about the grip of a user. The historical data can be used by a trend extraction engine of the portable computing device or remote device to track the grip strength of the user and/or find trends in the data provided by the pressure sensors.

For example, in some embodiments, various grip-related features may be fed into a trained machine learning model (e.g., regression model, neural network, deep learning network, batch or stochastic gradient descent, application of the normal equations, etc.), case-based reasoning algorithm, or other clinical reasoning algorithm to derive one or more grip metrics. In some embodiments, the model may be trained, for instance, using historical data related to the user and/or to a population of users. In some embodiments, the information used for deriving the grip metric may include or even be wholly limited to grip-related features or other information that may be captured by the one or more pressure sensors. In some embodiments, the information used for deriving the grip metric may alternatively or additionally include information such as information from a previous electronic medical record (EMR) of the user, information from wearable devices or other sensors carried by the user, information about family members or others associated with the user (e.g., family member EMRs), etc.

The portable computing device can notify the user that they are scheduled to perform a grip strength exercise, as well as provide details about their last grip strength exercise. During the grip strength exercise, the user grips the portable computing device for a period of time. Typically, the pressure applied to the portable computing device by the hand of the user may quickly climb to a maximum pressure and then decline for a remainder of the period of time. The portable computing device can then, for each finger and/or entire hand, record the time to maximum pressure, the maximum pressure, rate of decay from the maximum pressure, average pressure during exercise, and/or any other metric related to handgrip strength. Each metric can then be stored in the grip feature database and compared, by the trend extraction engine, against existing grip data in the grip feature database. If one or more of the metrics of the user has changed beyond some threshold, a notification can be provided to the user and/or an electronic healthcare system such as the user's doctor's office for analysis by a healthcare professional. The threshold can be a static or dynamic threshold that is set by a medical specialist, the user, and/or a research database in communications with the portable computing device.

In some embodiments, the grip metrics can include various scores that estimate a condition of a patient, and the condition can be incorporated into an EMR of the user. For example, the metrics can include a fatigue score that is based at least on a rate of decay of pressure. The fatigue score can provide insight into the progress of certain disease such as Parkinson's disease, and the fatigue score can be used to determine clinical pathways for treating Parkinson's disease. Any of the data gathered by the portable electronic device based on pressure applied by a user's grip can be used to make recommendations to the user regarding diagnosing and/or improving health conditions of the user. Furthermore, a graphical user interface (GUI) of the portable computing device can be used to provide images that convey trends of grip strength measurements over time. For example, the GUI can provide a plot of a trend of average or maximum grip strength over time. This can be helpful for athletes that want to track the progress of their handgrip exercises and/or user's with conditions where grip strength is affected.

In some embodiments, an electronic healthcare system can communicate with the portable computing device to issue exercise recommendations to the user. For example, a doctor can use an office computer to recommend a daily handgrip exercise to a patient. The portable computing device can provide daily notifications to a user in response to receiving a recommendation from the doctor and track the data generated during the daily handgrip exercises. The data can be communicated back to the office computer of the doctor so that the doctor can see the results of each exercise performed by the patient. Furthermore, a patient can be issued a notification when the portable computing device does not detect a handgrip exercise being performed for a period of time set by the user or the doctor. In some embodiments, the one or more pressure sensors of the portable computing device can be used to authenticate a user. For example, a user can grip the portable computing device for a period of time and the portable computing device can compare the grip data collected during the gripping period to previous grip data such as distance between fingers, grip strength, fatigue, and/or any other data. When the grip data is determined to be similar to the previous grip data, the portable computing device can authenticate the user and perform some operation (e.g., unlock) in response to authenticating the user.

Furthermore, the portable computing device can include a feedback module that can provide feedback to a user based on grip strength assessments made at the portable computing device. For example, the user can receive feedback from the feedback module when a grip strength exercise is completed and/or the results of the grip strength exercise compared to previous results from past exercises. Furthermore, the feedback module can communicate with an electronic healthcare system, such as an EMR database, and provide feedback regarding how the results of a grip strength exercise compare to information provided in the EMR database. Furthermore, data generated by the portable computing device as a result of a grip strength exercise can be incorporated into the EMR database.

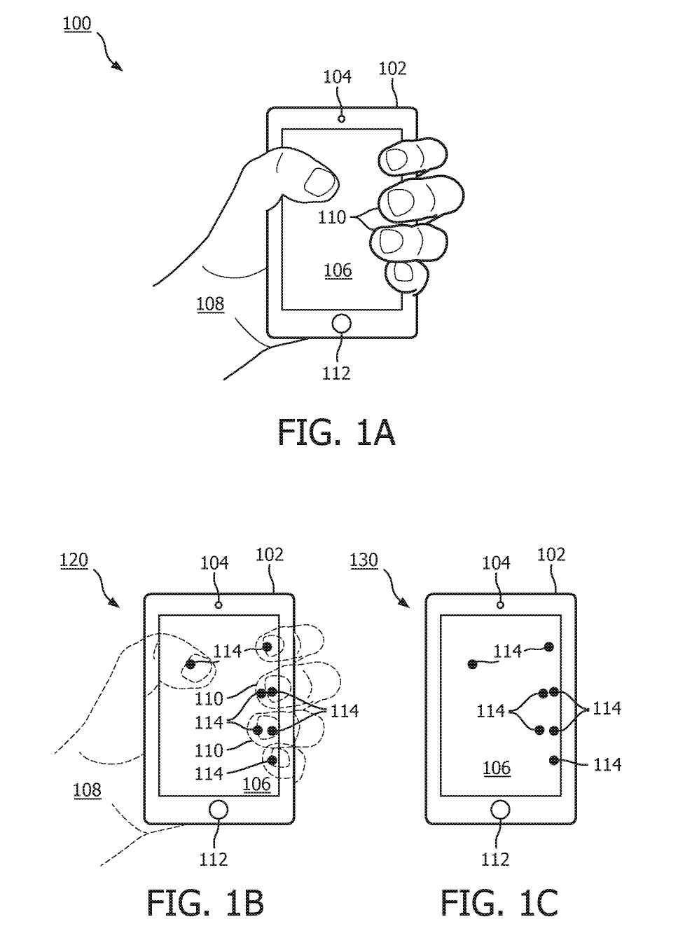

FIGS. 1A-1C illustrate operations that are performed when using a grip strength assessment application according to various embodiments. As provided in diagram 100 of FIG. 1A, the application can operate on a personal, e.g., mobile, computing device 102 that includes a touch-sensitive display 106, a microphone 112 and a speaker 104. The application can assess an amount of pressure applied by a hand 108 of a user instantaneously or over a period of time. The computing device 102 can determine an overall pressure applied by the user and/or individual pressures applied by each finger 110 of the user. Initially, at FIG. 1A, the user grips the computing device 102 with their hand 108 and wraps their fingers 110 around the device. The fingers 110 and/or hand 108 of the user can contact the touch-sensitive display 106 at different locations on the touch-sensitive display 106. Diagram 120 of FIG. 1B illustrates non-limiting examples of different points of contact 114 that can be identified by the computing device 102 using the touch-sensitive display 106. The points of contact 114 may correspond to all five fingers 110 of the hand 108 of the user, or less than five fingers. Each point of contact 114 can be associated with a different amount of pressure and a different location on the touch-sensitive display 106. Diagram 130 of FIG. 1C illustrates the example points of contact 114 without the hand 108 depicted

By pressing on the touch-sensitive display 106, the user can directly or indirectly influence one or more pressure sensors of the computing device 102. For example, the computing device 102 can include an array of pressure sensors that output signals that vary according to an amount of pressure applied at different locations of the touch-sensitive display 106. The signals from the array of pressure sensors can be used to calculate the pressure being applied by each finger 110. These pressure values can be used to calculate different metrics indicative of a condition of the user. For example, the computing device 102 can connect to an electronic healthcare system such as an electronic medical records (EMRs) database and compare the metrics to the results of previous handgrip strength assessments. Declining handgrip strength can be indicative of mortality and other diseases. Accordingly, if the user's handgrip strength is trending lower, the user or a device associated with a healthcare professional can be notified by the computing device 102 of the trend. In some embodiments, the pressure values from the grip of the user can be used to generate one or more metrics related to grip. The metrics can include overall grip strength, strength of individual fingers, absolute positions of fingers, distance between fingers, average grip or finger strength, time to peak pressure for the hand 108 and/or each finger, fatigue score, and/or any other metric related to grip.

One or more metrics derived from the pressure values can be compared to a static or dynamic threshold that can be based on data in a medical study, a value indicated by a healthcare professional, previous metrics generated by the computing device 102, and/or any other suitable data related to grip. If a metric satisfies the threshold, the computing device 102 can provide a notification to the user, a healthcare professional, and/or make changes to an EMR accessible to the computing device 102. In this way, a user or healthcare profession does not need to manually enter handgrip data manually into an EMR in order to track the handgrip data.

In some embodiments, user authentication can be performed when a user is gripping the computing device 102. For example, when the computing device 102 is in a locked state where access to applications is limited, a user can grip the computing device 102 with their hand 108. While the user is gripping the computing device 102, the computing device 102 can determine the pressure being applied to the computing device 102 by the hand 108 and/or individual fingers 110 of the user. Furthermore, the computing device 102 can determine the locations of the points of contact 114 and/or the distances between points of contact 114. In some embodiments, an overall shape created by the points of contact 114, as illustrated in FIG. 1C can be determined and used to authenticate the user. For instance, points of contact for a small child may be closer together than, say, those of an adult. Once the computing device 102 determines the pressure, points of contact 114, overall shape, and/or any other metric related to handgrip, the computing device 102 can compared the metric to a stored metric. If the determined metric is within a certain tolerance of the stored metric, the computing device 102 can authenticate the user and perform some operation based on the authentication (e.g., transition into an unlocked state, perform/record grip measurements, etc.).

In some embodiments, determining metrics can be performed by identifying the pressure values (pᵢ) corresponding to each point of contact 114, and/or a spatial information (xᵢ, yᵢ) related to each point of contact 114, where i=1, 2, . . . , n, and n is the total number of points of contact 114. A feature extraction module of the computing device 102 can use these values and information to calculate the different metrics related to handgrip. For example, an inner distance between touch points can be calculated using Equation (1) below, where i, j=1, 2, . . . , n, and n is the total number of points of contact 114.

$${d}_{i , j} = {( {( {x}_{i} - {x}_{j} )}^{2} + {( {y}_{i} - {y}_{j} )}^{2} )}^{\frac{1}{2}} \qquad ( 1 )$$

By calculating distance between points of contact 114, unique features can be identified relative to the grip of a user. For example, because of the unique bone structure of the hand 108 of the user, a point of contact 114 of the pinky finger of the user can be a closer distance to a point of contact 114 of a ring finger of the user when compared to other persons. This type of unique feature can be used to authenticate the user. Furthermore, identifying positions and/or distances between points of contact 114 can allow for more accurate overall pressure calculations. For example, a mechanical resistance exerted by the touch-sensitive display 106 may vary at different locations on the touch-sensitive display 106. Mechanical resistance at the center of the touch-sensitive display 106 may be lower than mechanical resistance at an edge of the touch-sensitive display 106. Identifying where each point of contact 114 is on the touch-sensitive display 106 can be used in combination with how much resistance is associated with the location of the point of contact 114 to more accurately determine the overall pressure of the handgrip. In some embodiments, the computing device 102 may store a lookup table that provides a correspondence between the locations on the touch-sensitive display 106 and the mechanical resistance associated with the locations.

Other metrics that can be calculated by the computing device can include overall grip strength, minimum pressure, maximum pressure, average pressure, and/or time to peak pressure. Overall grip strength can be calculated as a sum of the pressure identified at each point of contact 114, as provided in Equation (2) below, where n is the number of fingers 110 and Pᵢ is pressure at an individual finger 110.

$$Overall Grip Strength = {\sum }_{i = 1}^{n} \frac{{P}_{i}}{n} \qquad ( 2 )$$

Additionally, as discussed herein, a fatigue score can be calculated for a user after or during a handgrip strength assessment exercise using the computing device 102. During the exercise, a user can grip the computing device 102 for a period of time and the pressure exerted by the hand 108 and/or fingers 110 of the user can be recorded. An exponential function, such Equation (3) provided below, can be fitted to the recorded pressure data. For example, the fatigue score can be the coefficient b from the fitted exponential function.

p=a*e^{-bt} (3)

In some embodiments, the computing device 102 can determine a difference between extremities of a user. For example, the computing device 102 can determine whether a user is gripping the computing device 102 or stepping on the computing device 102. This difference can be determined by identifying the locations of points of contact 114 and comparing them to previously recorded locations of points of contacts that are stored in associated with an identifier for the extremity. The computing device 102 can use pressure sensors to determine a weight of the user when the user is stepping on the computing device 102. In this way, the computing device 102 can provide metrics that are based on the weight of the user and transmit weight related data to an electronic healthcare system. Additionally, the user will be able to track their weight on a regular basis from almost anywhere without the need to carry a separate weight scale device.

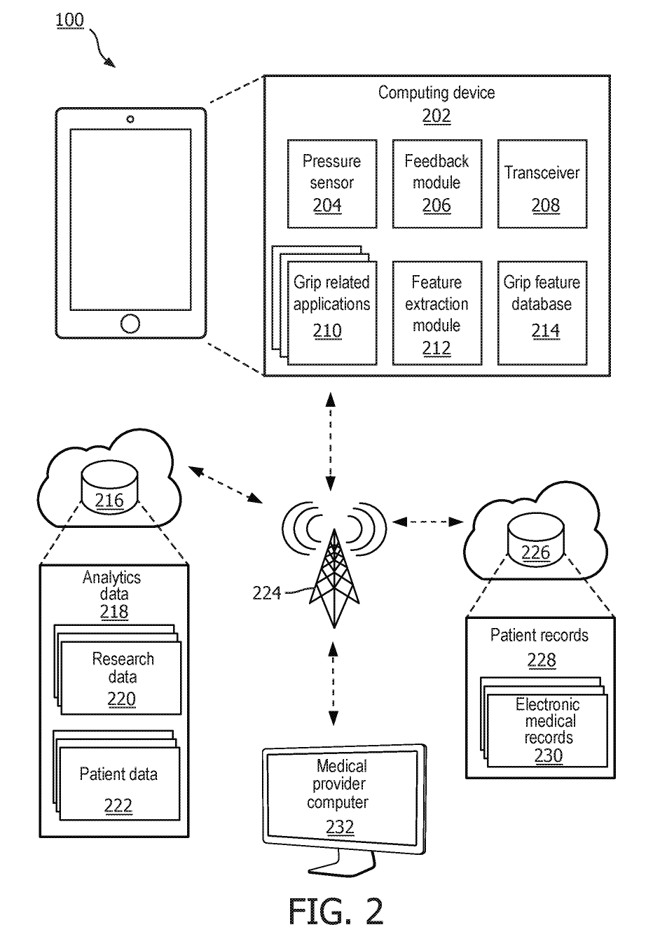

FIG. 2 illustrates a system diagram 200 of a computing device 202 that can collect, analyze, and share handgrip data with other computing devices, such as those associated with an electronic healthcare system. The computing device 202 can correspond to computing device 102, and/or any other device that can gather handgrip data as discussed herein. The computing device 202 can include one or more pressure sensors 204 for detecting pressure exerted by a user on the computing device 202. Additionally, the computing device 202 can include a feedback module 206 that provides feedback before, during, or after a handgrip exercise using the computing device 202. Feedback from the feedback module 206 can include sounds, light, vibrations, and/or any other suitable medium for providing feedback. For example, when a user is performing an exercise routine, the computing device 202 can detect whether the user performed a handgrip related exercise during the exercise routine. If the user did not perform a handgrip exercise during the exercise routine, the feedback module 206 can cause the computing device 202 to provide feedback to the user as a reminder to perform the handgrip related exercise. In some embodiments, the feedback module 206 can provide feedback to a user during a handgrip exercise. For example, the computing device 202 can store historical grip metrics in a grip feature database 214. The historical grip metrics can include a maximum grip pressure exerted by the user on the computing device 202 during a previous handgrip exercise. If the user exceeds the maximum grip pressure, the feedback module 206 can provide feedback as an indication that the user has achieved a new personal record. Alternatively, the user can be notified when their maximum grip pressure exerted during a particular exercise has declined below a threshold or outside of a tolerance of a previously recorded maximum pressure. Such a notification can be useful because the decline in maximum grip pressure can be an early indicator of a negative health condition.

The computing device 202 can also include a transceiver 208 for communicating grip related data to other devices over a network 224. The transceiver 208 can be one or more transceivers capable of communicating over a cellular network, Wi-Fi network, local area network, Bluetooth connection, near-field connection, and/or any other wired or wireless connection suitable for transmitting data. The computing device 202 can send handgrip data to an analytics server 216 that analyses various medical data for recommending clinical pathways. The analytics server 216 can process and/or store analytics data 218 that can include research data 220 and/or patient data 222. The patient data 222 can include the handgrip data provided by the computing device 202, as well as any other medical data that can be provided by the computing device 202. Furthermore, the analytics server 216 can access patient records 228 from a medical records database 226, which can store electronic medical records (EMRs) 230 for different patients. The analytics server 216 can use the data from the electronic medical records 230 in combination with research data 220 and patient data 222 in order to recommend clinical pathways toward treatment of a user. For example, the analytics server 216 can extract trends in the grip data provided by the computing device 202 and determine whether the trends are indicative of a potentially harmful medical condition. If the trends are indicative of a potentially harmful medical condition, the analytics server 216 can transmit data related to the medical condition to the medical records database 226 for updating a user's EMRs. The analytics server 216 can also transmit data related to the medical condition to the computing device 202 for notifying the user, or to a medical provider computer 232 associated with a medical provider of the user.

The computing device 202 can further include grip related applications 210 for providing different handgrip exercises to be completed by a user. The grip related applications 210 can include a disease specific application that is programmed to mitigate, through handgrip exercises, symptoms of certain diseases. For example, the grip related applications 210 can include an exercise application for users suffering from Parkinson's disease in order ensure that the user is regularly exercising their handgrip in order to provide some relief to the user. Furthermore, the grip related applications 210 can include an application for training athletes. The application can track the pressure exerted by a user during different exercises and issue new exercises for the user in order to ensure that the user is being challenged during their training.

The computing device can also include a feature extraction module 212 that can be used to derive the various grip metrics from the sensor data received from the pressure sensor 204, time data, finger location data, and/or any other source of data available to the computing device 202. The feature extraction module 212 can calculate grip strength parameters including overall grip strength, individual finger strength, finger positions, inner distances between points of contact of the fingers, time to peak pressure, fatigue score, and/or any other metric related to grip. Any of these metrics can be stored in the grip feature database 214 and/or transmitted to the analytics server 216, medical records database 226, the medical provider computer 232, and/or any other device capable of reading grip metric data. Furthermore, the feature extraction module 212 can also performed any of the operations that can be performed by the analytics server 216. For example, the feature extraction module 212 can identify trends in a user's health using data generated by the feature extraction module 212, the grip feature database 214, data provided in the electronic medical records 230, research data 220, and/or patient data 222.

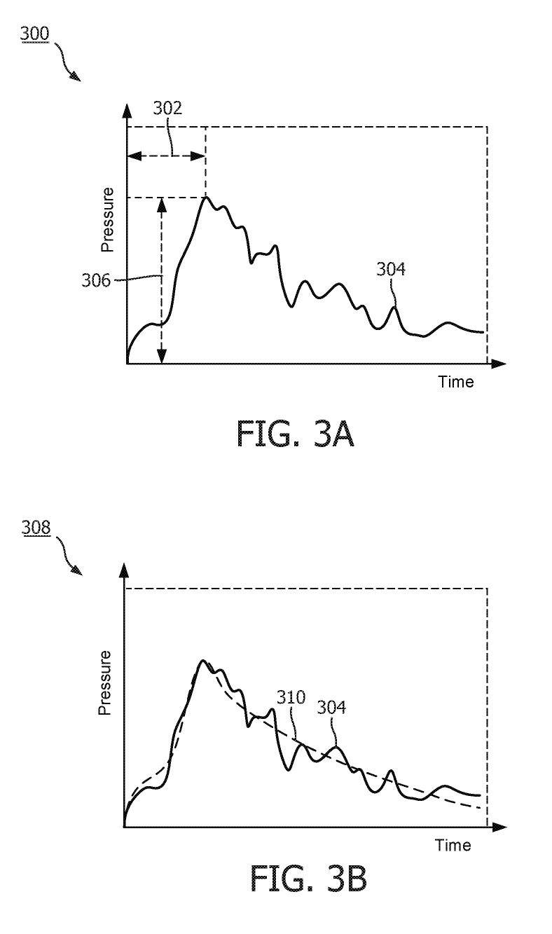

FIGS. 3A and 3B illustrate plots 300 and 308 of handgrip pressure recorded by a computing device over time. Specifically, FIG. 3A illustrates a plot 300 of pressure 304 exerted on a computing device, such as any of the devices discussed herein. The pressure 304 can be exerted during an exercise being performed by the user with the computing device to track various metrics related to handgrip. The computing device can use the pressure 304 data to identify a maximum pressure 306 during the exercise and a time 302 to maximum pressure 306. The maximum pressure 306 and/or the time 302 to maximum pressure 306 can be tracked for multiple exercises and analyzed to determine a trend in the maximum pressure 306 exerted by a user during the exercise. A declining trend for the time 302 to maximum pressure 306 and the maximum pressure 306 can be indicative of mortality and/or other medical conditions that may require medical treatment. Therefore, when such a trend is identified by the computing device, the computing device can notify a user or send trend data to a device associated with a medical provider responsible for the user.

FIG. 3B illustrates a plot 308 that includes a fitted exponential curve 310 for determining an exponential equation that best fits the plot of pressure 304. As discussed herein with respect to Equation (3), the decay of the exponential curve 310 from the maximum pressure 306 can be used to identify a fatigue score for the user. The fatigue score can be indicative of an amount of fatigue experienced by a user and can be used by medical providers in a variety of ways to treat patients. For example, a user that is suffering from Parkinson's disease can track their fatigue score. If their fatigue score over time indicates that they are experiencing more fatigue, the user will know to perform more handgrip exercises in order to mitigate the fatigue.

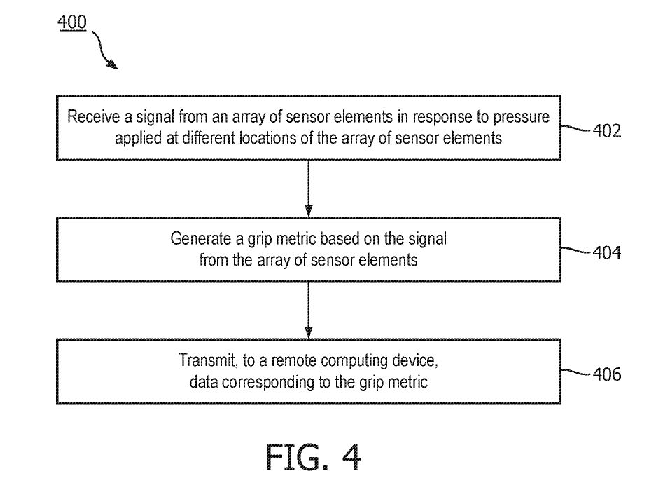

FIG. 4 illustrates a method 400 for transmitting handgrip data to a remote computing device, according to some embodiments. The method 400 can be performed by any computing device, controller, and/or apparatus discussed herein. As shown in FIG. 4, the method 400 begins at block 402, and involves the computing device receiving a signal from an array of sensor elements in response to pressure applied at different locations of the array of sensor elements. The sensor elements can be a capacitive touch sensing array disposed within the computing device and capable of being responsive to pressure applied to the display of the computing device. At block 404, the computing device generates a grip metric based on the signal from the array of sensor elements. The grip metric can include any of the metrics discussed herein and can be generated simultaneous to receiving the signal from the array. In this way, the computing device can provide a graphical user interface (GUI) that presents the grip metric simultaneous to the user applying pressure to the computing device. At block 406, the computing device transmits, to the remote computing device, data corresponding to the grip metric. The remote computing device can be associated with an electronic healthcare system that stores electronic medical records for the user.

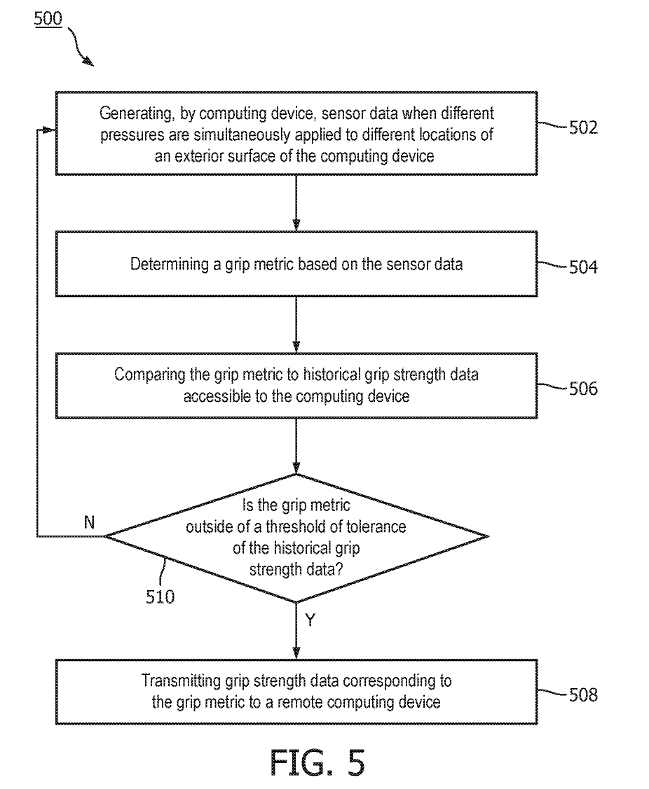

FIG. 5 illustrates a method 500 for comparing grip strength data and transmitting grip strength data based on the comparing, according to some embodiments. The method 500 can be performed by any computing device, controller, and/or apparatus discussed herein. As shown in FIG. 5, the method 500 begins at block 502, and involves the computing device generating sensor data when different pressures are simultaneously applied to different locations of an exterior surface of the computing device. At block 504, the computing device determines a grip metric based on the sensor data. At block 506, the computing device compares the grip metric to historical grip strength data accessible to the computing device. At block 510, a determination is made whether the grip metric is outside of a threshold of tolerance of the historical grip strength data. When the grip metric is outside of the threshold tolerance of the historical grip strength data, then, at block 508, the computing device transmits, to a remote computing device, grip strength data corresponding to the grip metric. When the grip metric is not outside of the threshold tolerance of the historical grip strength data, then block 502 is repeated subsequently when different pressures are simultaneously applied to different locations of an exterior surface of the computing device.

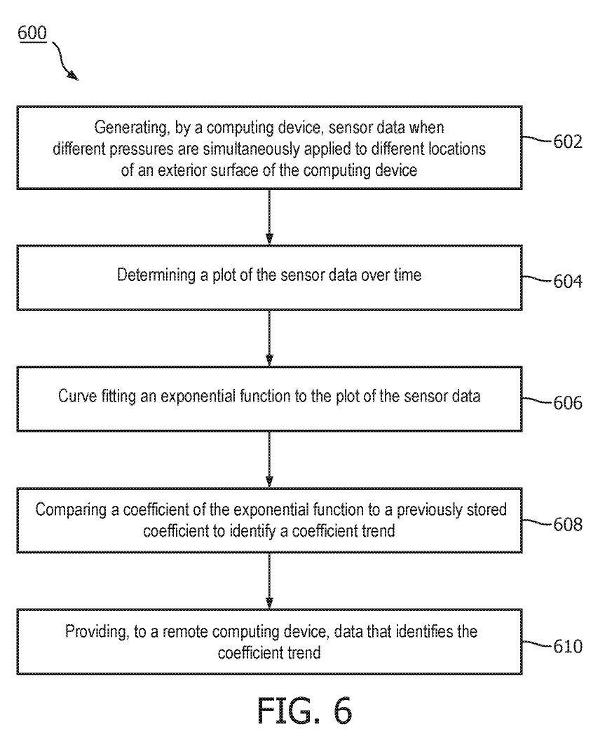

FIG. 6 illustrates a method 600 for generating and comparing coefficients that are derived from grip strength measurement data, according to some embodiments. The method 600 can be performed by any computing device, controller, and/or apparatus discussed herein. As shown, the method 600 begins at block 602, where the computing device generates sensor data when different pressures are simultaneously applied to different locations of an exterior surface of the computing device. At block 604, the computing device generates a plot of the sensor data over time. The sensor data can correspond to different amounts of pressure applied to the different locations of the exterior surface of the computing device or a total pressure applied to the different locations of the exterior surface of the computing device. At block 606, the computing device curve fits an exponential function to the plot of the sensor data. At block 608, the computing device compares a coefficient of the exponential function to a previously stored coefficient to identify a coefficient trend. The coefficient can be a coefficient of a power variable of the exponential function or a coefficient of a non-power variable of the exponential function. At block 610, the computing device provides, to a remote computing device, data that identifies the coefficient trend.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope

What is claimed is:

1. A computing device, comprising:
a pressure sensor comprising an array of sensor elements, the pressure sensor configured to output a signal in response to pressure applied at different locations of the array of sensor elements;
a display device, wherein the array of sensor elements are configured to receive the pressure in response to an initial force of pressure applied to the display device, and the signal is output in response to pressure applied at different locations on the display device;
one or more processors configured to generate a grip metric based on the signal from the pressure sensor; and
a communications interface configured to transfer data corresponding to the grip metric to a remote computing device.

2. The computing device of claim 1, wherein the grip metrics include a distance measurement between at least two points of contact at the display device.

3. The computing device of claim 1, wherein the grip metrics include a rate of decay of grip pressure over a period of time when the display device is receiving a variable force of pressure.

4. The computing device of claim 1, wherein the grip metric includes an overall grip strength measurement that is based on a total of individual forces of pressure at the different locations of the array of sensor elements.

5. The computing device of claim 1, wherein the communications interface is further configured to receive, from the remote computing device, analytical data based on the grip metric.

6. The computing device of claim 1, wherein the one or more processors are further configured to authenticate a user based on the signal from the pressure sensor.

7. A method for gathering and communicating grip strength data using a mobile computing device, the method comprising steps of:
by the mobile computing device:
generating sensor data when one or more pressures are simultaneously applied to different locations of a touch-sensitive display of the mobile computing device;
determining a grip metric based on the sensor data;
comparing the grip metric to historical grip metric data accessible to the computing device; and
when the grip metric is outside of a threshold tolerance of the historical grip metric data:
transmitting data corresponding to the grip metric to a remote computing device.

8. The method of claim 7, further comprising:
performing a curve fitting operation on the sensor data to identify an exponential function representing the sensor data;
wherein the grip metric includes a parameter of the exponential function.

9. The method of claim 8, wherein the grip metric is a maximum total pressure exerted on the touch-sensitive display of the mobile computing device, and comparing the grip metric to historical grip metric data includes comparing the maximum total pressure exerted to a historical value of maximum total pressure previously recorded by the mobile computing device.

10. The method of claim 8, wherein the sensor data is collected over a period of time and the grip metric includes a time to reach the maximum total pressure exerted.

11. The method of claim 8, wherein transmitting the data is performed simultaneous to generating the sensor data.

12. A non-transitory computer-readable medium configured to store instructions that when executed by one or more processors of a computing device, cause the one or more processors to perform steps that include:
receiving sensor data from an array of touch-sensitive elements of the computing device;
determining amounts of pressure exerted at different locations of the array of touch-sensitive elements;
determining a distance between the different locations of the array of touch-sensitive elements;
generating a grip strength metric using the amounts of pressure exerted and the distance between the different locations; and
providing an indication at a display of the computing device indicating whether the grip strength metric is different than a historical grip strength metric.

13. The non-transitory computer-readable medium of claim 12, wherein the steps further include:
accessing an electronic medical record (EMR) in a remote device accessible to the computing device, wherein the historical grip strength metric is stored in the EMR.

14. The non-transitory computer-readable medium of claim 12, wherein the steps further include:
modifying an exercise regimen managed by an exercise application on the computing device when the grip strength metric is lower than a historical grip strength metric.

15. The non-transitory computer-readable medium of claim 12, wherein the grip strength metric is based on an exponential function curve fitted to at least a portion of the sensor data.
